(19) United States
(12) Patent Application Publication
(10) Pub. No.: US 2008/0076746 A1
(43) Pub. Date: Mar. 27, 2008

(54) Method of Treating Fish and Crustaceans With a Tellurium Based Immunomodulator

(76) Inventors: Benjamin Sredni, Kfar-Saba (IL); Michael Albeck, Ramat-Gan (IL); Rami R. Avtalion, Ramat-Gan (IL); Eitan Okun, Ramat-Gan (IL)

Correspondence Address: Martin D. Moynihan;PRTSI, Inc., P.O. Box 16446, Arlington, VA, 22215, US

(21) Appl. No.: 10/582,296

(22) PCT Filed: Dec. 9, 2004
(86) PCT No.: PCT/IB04/04095

§ 371 Date: Apr. 20, 2007

Related U.S. Application Data
(60) Provisional application No. 60/529,142, filed on Dec. 12, 2003.

(51) Int. Cl. A61K 33/24; A61K 31/28; A61P 31/04; A61P 31/10

(52) U.S. Cl. 514/184; 514/492

(57) ABSTRACT

A method for the direct or adjunct treatment of bacterial or fungal infections in fish and crustaceans including shell fish which is based on providing an effective amount of an organic tellurium compound in an aqueous environment to which said fish or crustaceans are exposed.

### BACKGROUND

Fish hold many aspects of the immune system similar to those of higher vertebrates. Until now, a number of cytokines have been identified in biological assays on the basis of their functional similarity to mammalian cytokine activities or detected through their biological and/or antigenic cross-reactivity with mammalian cytokines (1). Recently, using computer-based tools, an IL-10 homologue was characterized in the puffer fish, (fugu rubripes) and predicted to have 183 amino acids (2). We recently characterized the IL-10 cytokine homologue in tilapia and carp using anti human mAb. SDS-PAGE results showed that IL-10L MW in both tilapia and carp is about 15 kDa. Such homology indicates that perhaps it also plays a major role in the fish immune system. AS101 (ammonium trichloro [dioxyethylene-O, O′] telurate) is a low molecular weight, non toxic compound that has been shown to have immune regulatory properties (3). Most of its activities have been primarily attributed to the direct inhibition of the anti-inflammatory cytokine IL-10, followed by the simultaneous increase of other cytokines, such as IL-1α, TNFα, IFNγ, IL-2, IL-12, and GM-CSF (3-6). We examined the effect of AS101 on intracellular levels of tilapia IL-10L in vitro and showed its ability to decrease intracellular IL-10L synthesis in a dose dependent manner. Stress response facilitates IL-10 production and secretion (7), which can cause immune suppression (8). In this study we showed that IL-10L secretion in vivo is up-regulated during stress reaction in fish, and that AS101 is able to decrease that secretion, without affecting the normal stress reaction as indicated by elevated serum glucose levels. Moreover, a protective effect of AS101 on stressed goldfish (Carassius auratus) infected with the opportunistic pathogen Aeromonas salmonicida was found. Interleukin-10 (IL-10) is known in mammalians to down-regulate the cellular immunity, resulting in increased susceptibility to opportunistic disease. The ability of the immunomodulator AS101 to down-regulate IL-10 levels has been shown in murines and humans. We have discovered an IL-10-like (IL-10L) cytokine in Tilapia and Carp fish using Western-Blot analysis with human IL-10 mAb and ELISA, and showed IL-10L kinetic expression of LPS stimulated cultures. We have also discovered that air-exposure stress resulted in the in vivo increase of serum IL-10L levels with the increase of blood glucose.

### SUMMARY OF THE INVENTION

Treatment of fish and crustaceans and in particular stressed fish by exposure to organic based tellurium compounds such as AS101 causes significant inhibition of IL-10L secretion to the serum, without affecting the normal stress reaction of the fish as expressed in increased glucose levels. Moreover stress induced Goldfish that were infected with Aeromonas salmonicida bacteria and treated with AS101 had significantly less wounds and mortality than control fish. Accordingly, the invention is directed to the direct or adjunct treatment of bacterial or fungal infections in fish and crustaceans including shell fish. The terms fish and crustaceans are used to include all species of fresh water and salt water fish including fish that are raised in fish farm environments or tropical pet fish as well as lobsters, crayfish, clams, oysters, shrimp, muscles and the like. The invention contemplates providing in the aqueous environment of the fish or crustaceans an amount of an organic tellurium compound that inhibits or treats an infection in fish or crustaceans. These amounts may vary but generally from 0.01 to 10 micrograms of the tellurium compound per ml of water may be employed. The invention also contemplates the concomitant administration of antibiotics, antifungal drugs in conventional doses along with an organic tellurium compound.

### BRIEF DESCRIPTION OF THE DRAWING

FIG. 1 discloses a dose-dependent inhibition of IL-10L by the immunomodulator AS101.

FIG. 2 discloses Serum IL-10L levels as measured prior to stress induction and at different intervals of time following stress induction

FIG. 3 discloses stress induction up-regulates IL-10L and glucose secretion, however, + only IL-10L is inhibited when stressed fish are treated with AS101.

FIG. 4 discloses AS101 treated fish infected with Aeromonas salmonicida show significantly less wounds (p=0.073) than untreated infected fish.

FIG. 5 discloses AS101 treated stressed fish infected with Aeromonas salmonicida show higher survival rates than control fish.

### DETAILED DESCRIPTION OF THE INVENTION

The organic tellurium compounds for use in the invention include those of the formula:

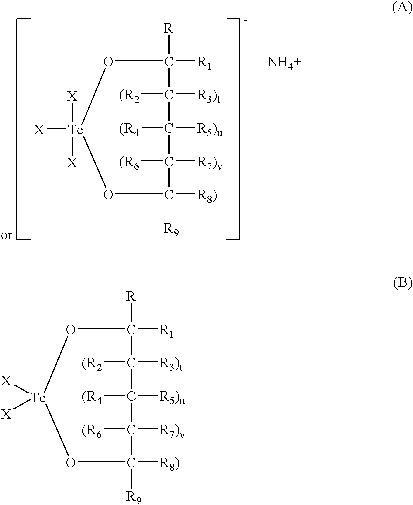

or

TeO₂or complexes of TeO₂ (C)

or

PhTeCl₃ (D)

or

TeX₄, when X is Cl, Br or F

or

(C₆H₅)₄P+(TeCl₃(O₂C₂H₄))— (E)

wherein t is 1 or 0; u is 1 or 0; v is 1 or 0; R, R₁, R₂, R₃, R₄, R₅, R₆, R₇, R₈, and R₉ are the same or different and are independently selected from the group consisting of hydrogen, hydroxyalkyl of 1 to 5 carbons, hydroxy, alkyl or from 1 to 5 carbon atoms, halogen, haloalkyl of 1 to 5 carbon atoms, carboxy, alkylcarbonylalkyl of 2 to 10 carbons, alkanoyloxy of 1 to 5 carbon atoms, carboxyalkyl of 1 to 5 carbons atoms, acyl, amido, cyano, amidoalkyl of 1 to 5 carbons, N-monoalkylamidoalkyl of 2 to 10 carbons, N,N-dialkylaminoalkyl of 4 to 10 carbons, cyanoalkyl of 1 to 5 carbons alkoxy of 1 to 5 carbon atoms, alkoxyalkyl of 2 to 10 carbon atoms and —COR₁₀ wherein R₁₀ is alkyl of 1 to 5 carbons; and X is halogen; while the ammonium salt is illustrated, it is understood that other pharmaceutically acceptable salts such as K+ are within the scope of the invention. The compounds with the five membered rings are preferred.

As used herein and in the appended claims, the term alkyl of 1 to 5 carbon atoms includes straight and branched chain alkyl groups such as methyl; ethyl; n-propyl; n-butyl, and the like; the term hydroxyalkyl of 1 to 5 carbon atoms includes hydroxymethyl; hydroxyethyl; hydroxy-n-butyl; the term halkoalkyl of 1 to 5 carbon atoms includes chloromethyl; 2-iodoethyl; 4-bromo-n-butyl; iodoethyl; 4-bromo-n-pentyl and the like; the term alkanoyloxy of 1 to 5 carbon atoms includes acetyl, propionyl, butanoyl and the like; the term carboxyalkyl includes carboxymethyl, carboxyethyl, ethylenecarboxy and the like; the term alkylcarbonylalkyl includes methanoylmethyl, ethanoylethyl and the like; the term amidoalkyl includes —CH₂CONH₂; —CH₂CH₂CONH₂; —CH₂CH₂CH₂CONH₂ and the like; the term cyanoalkyl includes

—CH₂CN; —CH₂CH₂CN; —CH₂CH₂CH₂CN and the like; the alkoxy, of 1 to 5 carbon atoms includes methoxy, ethoxy, n-propoxy, n-pentoxy and the like; the terms halo and halogen are used to signify chloro, bromo, iodo and fluoro; the term acyl includes R₁₆CO wherein R₁₆ is H or alkyl of 1 to 5 carbons such as methanoyl, ethanoyl and the like; the term aryl includes phenyl, alkylphenyl and naphthyl; the term N-monoalkylamidoalkyl includes —CH₂CH₂CONHCH₃,
—CH₂—CONHCH₂CH₃; the term N,N-dialkylaminoalkyl includes —CH₂CON(CH₃)₂; CH₂CH₂CON(CH₂—CH₃)₂. The tellurium based compounds that are preferred include those of the formula:

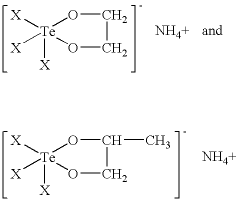

wherein X is halogen. The preferred halogen species is chloro.

Other compounds which are based on tellurium and may be used in the practice of the invention include PhTeCl₃, TeO₂ and TeX₄ (C₆H₅)₄ P+(TeCl₃ (O₂C₂H₄))— (Z. Naturforsh, 36, 307-312 (1981). Compounds of the following structure are also included:

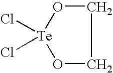

Other compounds useful for the practice of invention include:

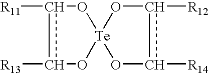

wherein R₁₁, R₁₂, R₁₃ and R₁₄ are independently selected from the group consisting of hydrogen, hydroxy-alkyl of 1-5 carbons atoms, hydroxy and alkyl of 1-5 carbons atoms.

Useful dihydroxy compounds for use in the preparation of compounds of structure A or B, include those of formula I wherein R, R₁, R₄ and R₅ are as shown in the Table:

TABLE

| | |
|---|---|
| | (I) |
| 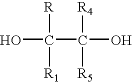 | |

| R | R₁ | R₄ | R₅ |
|---|---|---|---|
| H | H | H | H |
| H | Cl | H | H |
| H | OCH₃ | H | H |
| H | COOCH₃ | H | H |
| H | H | CN | H |
| H | CHO | H | H |
| H | H | COOH | H |
| H | CH₂COOH | H | H |
| H | H | CH₂COOCH₃ | H |
| H | I | H | H |
| H | H | Br | H |
| H | H | CONH₂ | H |
| H | H | CH₂OH | H |
| H | COOH | H | H |

Other dihydroxy compounds for use in the preparation of compounds A and B include those of formula II wherein R, R₁, R₂, R₃, R₄ and R₅ are as shown in the Table:

| | |
|---|---|
| | (II) |
| 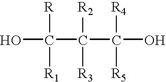 | |

| R | R₁ | R₂ | R₃ | R₄ | R₅ |
|---|---|---|---|---|---|
| H | H | H | H | H | H |
| H | H | Cl | H | H | H |
| H | CH₂OH | H | H | H | H |
| H | H | OH | H | H | H |
| H | H | H | CH₃ | H | H |
| H | H | H | CH₂Cl | H | H |
| H | H | H | COOH | H | H |
| H | H | H | CH₂COOH | H | H |
| H | H | H | CHO | H | H |
| H | H | H | H | H | CH₂CHO |
| H | H | CONH₂ | H | H₂ | CH₃ |
| H | H | H | CN | H | H |
| H | H | H | H | CH₂COHN₂ | H |
| H | H | H | COOCH₃ | H₃ | H |
| H | H₃ | OCH₃ | H | H | H |

Other dihydroxy compounds for use in making compound of formula A and B include those of formula III wherein R, R₁, R₂, R₃, R₄ and R₅ are as shown in the Table.

| | | | | | | | (III) |
|---|---|---|---|---|---|---|---|
| 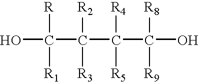 | | | | | | | |
| R | R₁ | R₂ | R₃ | R₄ | R₅ | R₈ | R₉ |
| H | H | H | H | H | H | H | H |
| H | H | Cl | H | H | H | H | H |
| H | H | H | H | Br | H | H | H |
| H | H | OCH₃ | H | H | H | H | H |
| H | H | CONH₂ | H | H | H | H | H |
| H | Br | H | H | H | H | H | H |
| H | H | H | H | CH₂COOH | H | H | H |
| H | H | Cl | Cl | H | H | H | H |
| H | CH₂COOH | H | H | H | H | H | H |
| H | H | CH₃ | H | H | H | H | H |
| H | CH₃ | H | H | H | H | H | H |
| H | CH₂Cl | H | H | H | H | H | H |
| H | H | H | I | H | H | H | H |
| H | CH₂CN | H | H | H | H | H | H |
| H | H | H | H | CH₂CH₂OH | H | H | H |

Additional dihydroxy compounds include those of formula IV wherein R, R₁, R₂, R₃, R₄ and R₅ are as shown in the Table.

| | |
|---|---|
| | (IV) |
| 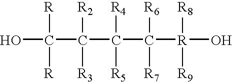 | |

| R | R₁ | R₂ | R₃ | R₄ | R₅ | R₆ | R₇ | R₈ | R₉ |
|---|---|---|---|---|---|---|---|---|---|
| H | H | H | H | H | H | H | H | H | H |
| H | H | Cl | H | H | H | Cl | H | H | H |
| H | H | Cl | Cl | H | H | H | H | H | H |
| H | H | CONCH₃ | H | H | H | Br | H | H | H |
| H | H | Br | H | H | H | CON(CH₃)₂ | H | H | H |
| H | H | H | OCH₃ | H | H | H | H | H | H |
| H | H | H | H | OCH₃ | H | H | H | H | H |
| H | H | H | H | CH₂COOH | H | H | H | H | H |
| H | H | COOH | H | H | H | H | H | H | H |
| H | CH₃ | H | H | H | H | H | H | H | H |
| CH₃ | H | H | H | H | CH₃ | H | H | H | H |
| H | CH₂CH₃ | H | H | H | H | H | Cl | H | H |
| H | CH₂CN | H | H | CH₂OH | H | H | H | H | H |
| H | H | H | I | H | H | H | H | CN | H |
| H | CH₂CH₂COOH | H | H | H | H | H | H | H | H |
| H | H | CHO | H | H | H | H | H | H | H |
| H | H | H | F | H | H | H | H | H | H |

Compounds of the following formula are also included:

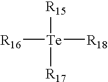

herein R₁₅, R₁₆, R₁₇ and R₁₈ are independently selected from halogen, alkyl of 1-5 carbons; aryl, acyl of 1-5 carbon hydroxyalkyl of 1-5 carbons and aminoalkyl of 1-5 carbons may be made by reacting the appropriate di, tri or tetrahalotelluride with the appropriate hydroxy compound which may be of the formula: HO—R₁₉;
wherein R₁₉; is alkyl of 1 to 5 carbons, haloalkyl of 1 to 5 carbons, aryl, alkylaryl, alkylamido of 1 to 5 carbons, alkylcarbonyl of 1 to 5 carbons, cyanoalkyl of 1 to 5 carbons, cyanoalkyl of 1 to 5 carbons, and an alkoxyalkyl of 2 to 10 carbons. Specific examples of R₁₆ include methyl, ethyl, n-propyl, phenyl, tolyl, amidoethyl, cyanomethyl, methyloxymethyl and CH₂CH₂COOH.

These compounds are described in United States Letters Patent No. 4,761,490 which is incorporated by reference. In addition, TeCl₄; TeBr₄ and compounds which give in aqueous solution TeO₂ preferably in the form of a complex such as for example TeO₂ complex with citric acid or ethylene glycol.

The preferred compound is ammonium trichloro (dioxoethylene-O,O′) tellurate.

### Treatment of Fish

Tilapia hybrids (Oreochromis niloticus X O. aureus) of 100-200 g in weight were raised in the Fish Immunology & Genetics Laboratory, Bar Ilan University. Carp (Cyprinus carpio) of 200-350 gr were purchased from the “Mevo-Hamah” farm (Israel). The fish were kept in 400 L plastic tanks with re-circulating water system. Temperature and oxygen levels were kept constant at 26±2° C. and 4-6 ppm, respectively. The term “organic tellurium” is defined to mean any tellurium element bonded to an organic moiety, including via atoms that differ from carbon, such as oxygen.

For in-vitro studies, AS101 was supplied as PBS solution, pH 7.4, at 4° C. For in-vivo experiments, AS101 powder was dissolved in the appropriate concentration in re-circulating water.

### Cell Culture

PBL (peripheral blood leukocytes) were isolated from heparinized blood on a ficoll paque bed (Amersham Bioscience) and cultured as previously reported (9). For AS101 treatments, AS101 was added directly to the cultures at final concentration of 0.025 to 25 μg/ml, preferably 0.05 to 0.5 μg/ml.

### Stress Induction

Fish were subjected to air exposure stress every 60 min for 90 sec during 4 hours (10). The fish were then divided into groups of 3, each separately held in 40 L plastic containers at 28°±2° C.

### Western Blot Analysis

PBL were lysed as previously reported (6). Samples were electrophoresed on 12% SDS-PAGE and blotted with anti-human-IL-10 mAb (Santa Cruz Biotechnology, Inc sc-8438) (1:660 dilution) and HRP-conjugate secondary Ab (Jackson Immuno-Research) (1:6600 dilution).

### Serum IL-10L Detection

Fish blood was coagulated during 2 hours at room temperature, then centrifuged for 10 min at 1800×g. IL-10L levels were quantified in the collected sera using ELISA (Human Interleukin-10 ELISA Kit, pierce-endogen) according to manufacturer's instructions.

### Stress Intensity Control

Blood samples were taken 1 hour after stress induction, sera separated by centrifugation as above and glucose levels were measured using Glucose TRINDER reagent (Sigma) according to manufacturers instructions, in a 96 microwell plate at 490 nm.

### Results

AS101 effect on the intracellular IL-10L levels of tilapia cell cultures

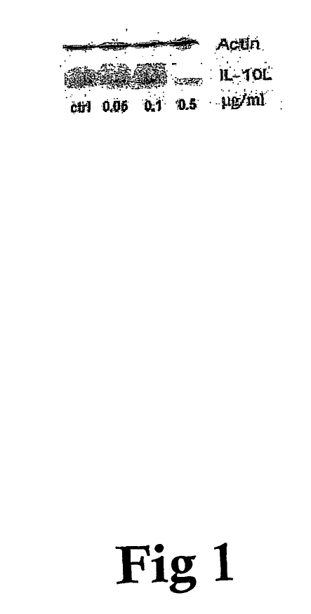

AS101 caused significant inhibition of intracellular tilapia IL-10L synthesis. This inhibitory effect was dose-dependant and complete inhibition was achieved with 0.5 μg/ml AS101 (FIG. 1).

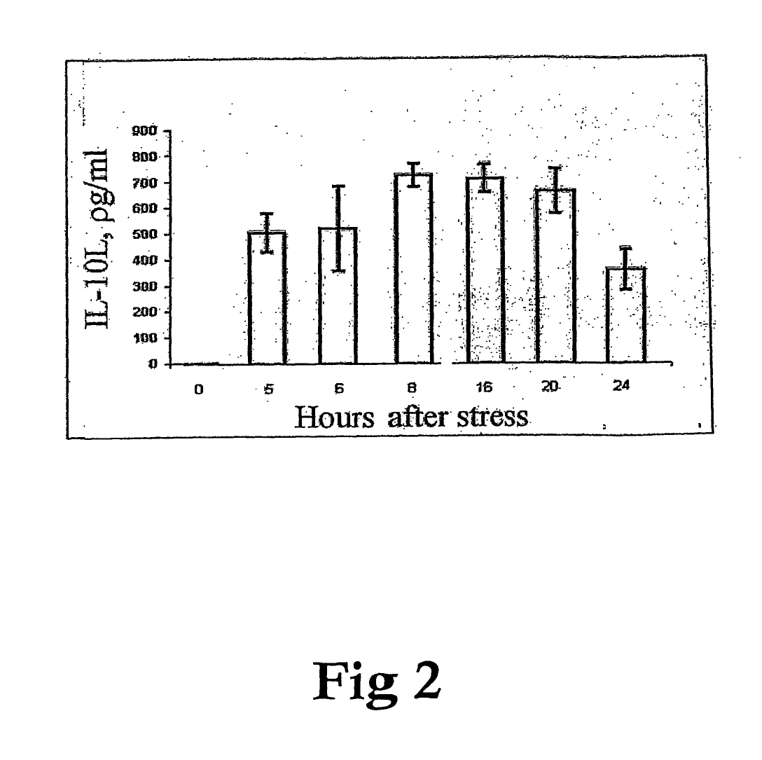

Influence of extensive stress on serum IL-10L levels Serum IL-10L levels were measured prior to stress induction and at different intervals of time (1, 5, 6, 8, 16, 20, and 24 Hrs) following stress induction. Significant IL-10L increase (p=0.001) started at 1 hour post stress, peaked on hours 8-16 and underwent significant decrease (p=0.006) afterwards (FIG. 2). AS101 effect on stress induced serum IL-10L and glucose levels

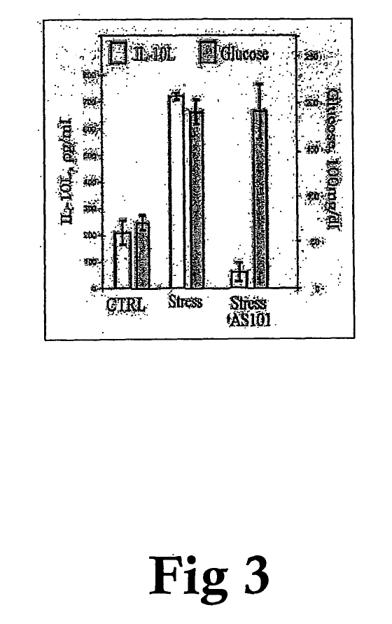

The maximal AS101 effect on IL-10L level (decrease from 719 to 209 pg/ml) was obtained 2 hours post-stressed following bath immersion in a 20-ppm water solution of this compound. AS101 treatment, while affecting IL-10L, had no effect on blood glucose levels which remained high (190 mg/dl±0.12) as in control stressed fish, (FIG. 3)

AS101 effect on stressed fish infected with Aeromonas salmonicida

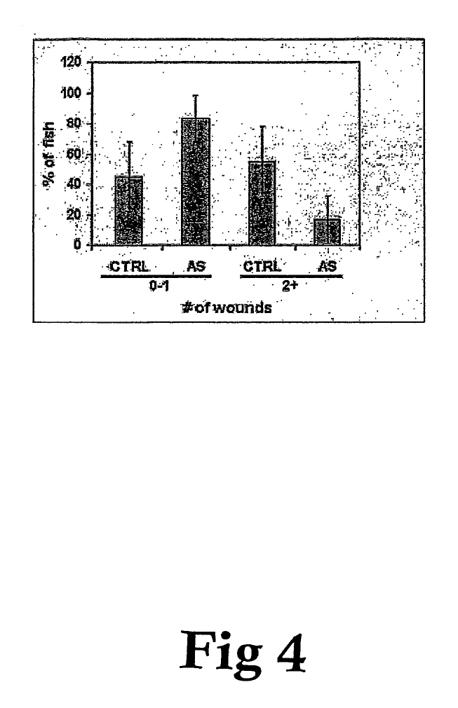

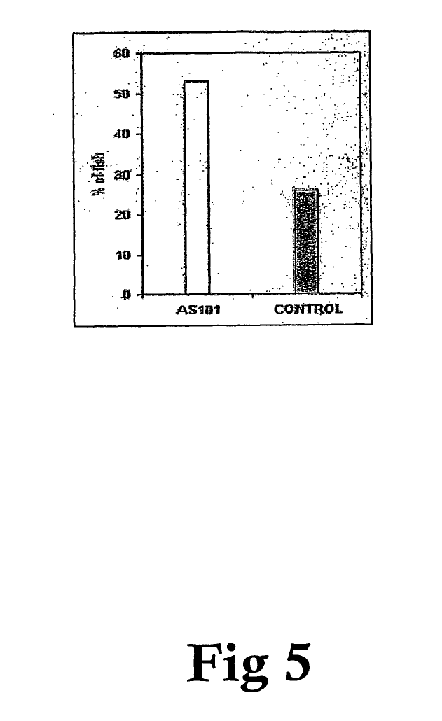

Stress induced fish that were immersed in soluble AS101 after being exposed to Aeromonas salmonicida had significantly lower number of wounds per fish (FIG. 4) and higher rate of survival (FIG. 5) in contrast to stressed fish infected with the bacteria and not treated with AS101. AS101 inhibits tilapia intracellular IL-10L in vitro in a dose dependant manner. The possibility that the inhibition was due to cell toxicity, was examined Blue staining, and did not show elevated cell death beyond control in all AS101 doses, indicating that AS101 has no which were taken 6 hours after stress induction and bath immersion in different AS101 concentrations showed a significant IL-10L decrease, mainly with AS101 dose of 20 μg/ml which was the most efficient. These results show that AS101 reached the fish blood probably by penetrating through the skin, the gills or the gut epithelia. Interestingly, both IL-10L and glucose, which showed significant blood elevation following stress induction, only IL-10L, was down regulated by the AS101 treatment.

These results suggest that the protective role of AS101 against Aeromonas salmonicida infections in stressed goldfish involves AS101 mediated IL-10L inhibition, emphasizing it as an effective agent against stress-induced immune suppression.

References which are incorporated into this application:
[1] Takashi Y, Teruyuki N. Interaction between the endocrine and immune Systems in Fish. Int Rev Cytol. 220:35-92; 2002
[2] Jun Z, Melody S C, Secombes C J. Characterisation, expression and promoter analysis of an interleukin 10 homologue in the pufferfish (Fugu rubripes). 55(5): 325-35; August 2003
[3] Sredni B, Caspi R R, Klein A, Kalechman Y, Danziger Y, Ben Ya'akov, M Tamari T, Shalit F, Albeck M. A new immunomodulating compound (AS-101) with potential therapeutic application. Nature. 330(6144): 173-176; November 1987
[4] Strassmann G, Kambayashi T, Jacob C O, Sredni B. The immunomodulator AS-101 inhibits IL-10 release and augments TNF alpha and IL-1 alpha release by mouse and human mononuclear phagocytes. Cell. Immunol. 176: 180; 1997.
[5] Kalechman Y, Zuloff A, Albeck M, Strassmann G, Sredni B. Role of endogenous cytokines secretion in radioprotection conferred by the immunomodulator ammonium trichloro (dioxyethylene-0-0′) tellurate Blood. 85: 1555; 1995
[6] Kalechman Y, Longo D L, Catane R, Shani A, Albeck M, Sredni B. Synergistic anti-tumoral effect of paclitaxel (Taxol)+AS101 in a murine model of B16 melanoma: association with ras-dependent signal-transduction pathways Int. J. cancer. 86: 281; 2000
[7] Elenkov I J and Chrousos G P. Stress Hormones, Proinflammatory and antiinflammatory Cytokines, and Autoimmunity. Ann. N.Y. Acad. Sci. 966: 290-303; 2002
[8] Akdis C A, Blaser K. Mechanisms of interleukin-10-mediated immunesuppression. 103 (2): 131-6; June 2001
[9] Rosenberg-Wiser S, and Avtalion R R. The cells involved in the immune response of fish. III. Culture requirement for phytohemagglutinin stimulated peripheral carp lymphocytes. Devel. Comp. Immunol. 6:693-702; 1982
[10] Melamed O, Timan B, Avtalion R R and Noga E J. Design of a stress model in the hybrid bass (Morone saxatilis x Morone Chrysops). Isr. J. Aguac-Bamidgeh. 51(1): 10-16; 1999

1. A method for the direct or adjunct treatment of bacterial or fungal infections in fish and crustaceans including shell fish which comprises providing an effective amount of an organic tellurium compound in an aqueous environment to which said fish or crustaceans are exposed.

2. A method for the direct or adjunct treatment of bacterial or fungal infections in fish and crustaceans as defined in claim 1 wherein the tellurium compound is selected from compounds of the formula: 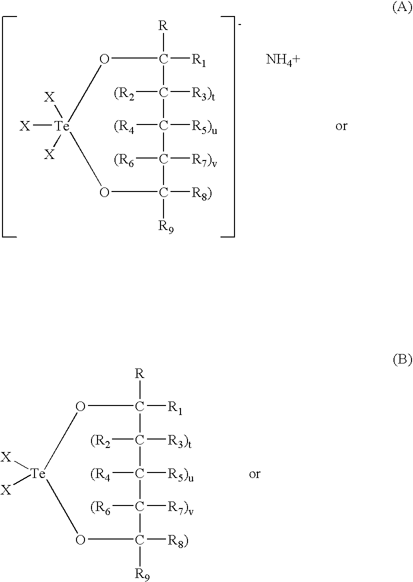
TeO₂or complexes of TeO₂ (C)
or
PhTeCl₃ (D)
or
TeX₄, when X is Cl, Br or F
or
(C₆H₅)₄P+(TeCl₃(O₂C₂H₄))— (E)
wherein t is 1 or 0; u is 1 or 0; v is 1 or 0; R, R₁, R₂, R₃, R₄, R₅, R₆, R₇, R₈, and R₉ are the same or different and are independently selected from the group consisting of hydrogen, hydroxyalkyl of 1 to 5 carbons, hydroxy, alkyl or from 1 to 5 carbon atoms, halogen, haloalkyl of 1 to 5 carbon atoms, carboxy, alkylcarbonylalkyl of 2 to 10 carbons, alkanoyloxy of 1 to 5 carbon atoms, carboxyalkyl of 1 to 5 carbons atoms, acyl, amido, cyano, amidoalkyl of 1 to 5 carbons, N-monoalkylamidoalkyl of 2 to 10 carbons, N,N-dialkylaminoalkyl of 4 to 10 carbons, cyanoalkyl of 1 to 5 carbons alkoxy of 1 to 5 carbon atoms, alkoxyalkyl of 2 to 10 carbon atoms and —COR₁₀ wherein R₁₀ is alkyl of 1 to 5 carbons; and X is halogen.

3. A method for the direct or adjunct treatment of bacterial or fungal infections in fish and crustaceans as defined in claim 1 wherein the tellurium compound is ammonium trichloro (dioxoethylene-O, O′) tellurate.

4. A method for the direct or adjunct treatment of bacterial or fungal infections in fish and crustaceans as defined in claim 1 where the treatment of bacterial or fungal infections in fish and crustaceans is a direct treatment.

5. A method for the direct or adjunct treatment of bacterial or fungal infections in fish and crustaceans as defined in claim 1 where the treatment of bacterial or fungal infections in fish and crustaceans is an adjunct treatment.

6. A method for the direct or adjunct treatment of bacterial or fungal infections in fish and crustaceans as defined in claim 1 where the treatment of bacterial or fungal infections in fish and crustaceans is based on the addition of 0.025 to 25 μg/ml to the aqueous environment of the fish or crustacean.
